(19) United States
(12) Patent Application Publication
(10) Pub. No.: US 2021/0219794 A1
(43) Pub. Date: Jul. 22, 2021

(54) Health and Wellness Monitoring System with a Handwashing Fixture

(71) Applicant: Hall Labs, LLC, Provo, UT (US)

(72) Inventor: David R. Hall, Provo, UT (US)

(21) Appl. No.: 17/153,858

(22) Filed: Jan. 20, 2021

Related U.S. Application Data
(60) Provisional application No. 62/963,428, filed on Jan. 20, 2020.

(51) Int. Cl. A47K 10/48; A61B 5/00

(52) U.S. Cl. CPC A47K 10/48; A61B 5/0205; A61B 5/6887

(57) ABSTRACT

A health and wellness monitoring system includes a hand washing fixture, such as a faucet or dryer, and a sensor, such as an imaging sensor, associated with the hand washing fixture and configured to acquire data from a user's hands.

### CROSS-REFERENCE TO RELATED APPLICATIONS

This application claims priority to U.S. Provisional Patent Application No. 62/963,428 titled “Apparatus for Imaging Hands for Detecting Health and Wellness Indicators” filed on 20 Jan. 2020, which is incorporated herein by reference in its entirety.

### TECHNICAL FIELD

The present disclosure relates to sink fixtures. More particularly, it relates to diagnostic sink fixtures equipped to provide health and wellness information to the user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Bathrooms present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Examining and monitoring the fingers and hands allows for the detection of health and wellness indicators related to several serious medical issues. For example, digital clubbing, also known as finger clubbing, is a known indicator of serious illness. Digital clubbing occurs in several stages that can be detected. “Digital Clubbing,” Malay Sarkar et al., Lung India, 2012 Oct.-Dec. 29(4), pp. 354-62, discusses in detail the stages of digital clubbing and their diagnostic significance. They provide lists of conditions that can cause digital clubbing. The entire disclosure of Sarkar is included herein by reference.

Computer Vision (“CV”) is a field of artificial intelligence that involves computers learning to view, interpret, and understand the visual world. It uses images from digital cameras and learning algorithms, computers use image processing to view multiple images and learn how to accurately recognize, identify, and classify object. CV is used in a variety of fields to identify things such as manufacturing defects, counterfeit currency, and early signs of disease in plants. Computer vision can be used to extract information from digital images to make decisions and take actions.

### SUMMARY

In a first aspect, the disclosure provides a monitoring system comprising a sink faucet fixture comprising a neck; a spout; and a sensor positioned to acquire data on a user's hands.

In a second aspect, the disclosure provides a hand dryer comprising at least one air dryer; and a sensor positioned to acquire data on a user's hands.

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a bottom perspective view of a first exemplary embodiment of a hand sensor incorporated into a faucet and hand dryer according to the present disclosure.

FIG. 2A is a isometric view of a second exemplary embodiment of a hand sensor incorporated into a hand dryer according to the present disclosure.

FIG. 2B is a side cross-sectional view of the embodiment of FIG. 2A.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, the term “faucet” is meant to refer to any device or mechanism for supplying water to a sink, basin, tub, or similar structure.

As used herein, the term “hand dryer” is meant to refer broadly to any device or mechanism for automating the process of drying hands, including air dryers.

As used herein, the term “user” refers to any individual who interacts with the faucet.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, transducers, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “imaging sensor” is meant to refer to any device for detecting and/or measuring a property of a person or of a substance that relies on electromagnetic radiation of any wavelength (e.g., visible light, infrared light, x-ray) or sound waves (e.g., ultrasound) to view the surface or interior of a user or substance. The term “imaging sensor” does not require that an image or picture is created or stored even if the sensor is capable of creating an image.

As used herein, the term “data connection” and similar terms are meant to refer to any wired or wireless means of transmitting analog or digital data and a data connection may refer to a connection within a toilet system or with devices outside the toilet.

As used herein, the term “processor” refers to logic circuitry that processes data from the imaging sensors, applies algorithms such as computer vision to analyze the images for health and wellness indicators, and provide the resulting data to users. The processor may also be used to perform other tasks such as analyzing image data to determine the identity of a user.

As used herein, the term “sensor array” is used to describe the combination of two or more sensors of one or more types in a scanning device.

### Exemplary Embodiments

The present disclosure relates to devices for analyzing hands and wrists for the purpose of identifying health malady indicators. More specifically, the present disclosure relates to the implementation of the analytical devices in handwashing and hand drying systems. This gives the benefit of frequent monitoring without visiting a medical professional. The present disclosure is related to a device for looking at fingernails, blood veins, wrinkles, sores, dryness, cracked bones, and tracking day-to-day changes. It also can measure and track hand temperature. The present disclosure relates to a system or device that uses imaging sensors, such as but not limited to visual, infrared, thermal, near infrared, and 3D cameras, to monitor hand health and wellness.

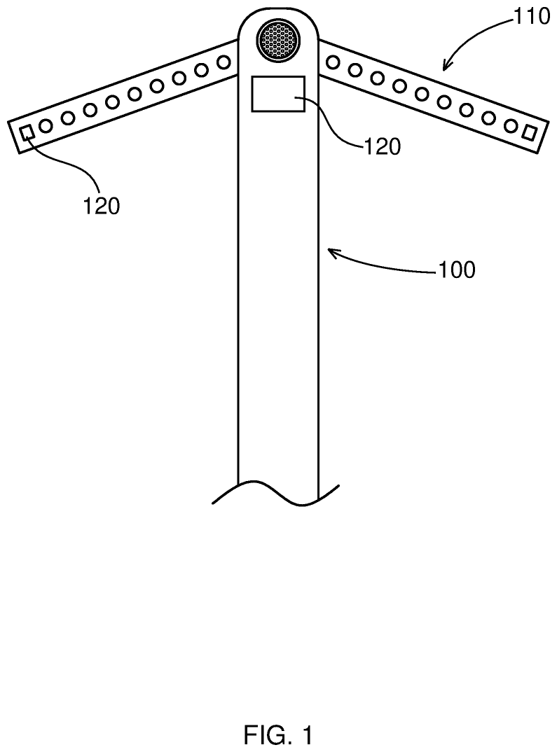

Now referring to FIG. 1, a combination faucet and hand dryer is shown. The faucet 100 extends out over a sink. Two hand dryer wands 110 extend substantially laterally to each side of the faucet 100. They include outlets 111 for air flow to blow downward along their length. A user dries his/her hands by placing the hands below the dryers 110 with their fingers extended. The user's fingers are typically extended straight out and held slightly apart from each other to allow for more effective drying. This creates an optimal opportunity for examining the hands and fingers.

In various exemplary embodiments, the hand dryer wands each contain one or more sensors 120. The sensors are placed on the underside of the wands 110 and/or faucet 100 and focus on the area where the hands are placed for drying. In various exemplary embodiments, one or more sensors 120 are placed on the underside or sides of the faucet and focused on the location where the hands are placed for drying. In a preferred embodiment, sensors 120 are located on both the faucet 100 and the wands 110.

In various exemplary embodiments, the sensors include one or more imaging sensors. The imaging sensors may include one or more of ultraviolet light sensors, infrared light sensors, near infrared light sensors, and visible light sensors. These sensors can detect hand size or shape, discoloration, swelling, veins, and changes over time across multiple uses. The images can also be used to identify a user and track hand appearance over time to detect trends relevant to health and wellness.

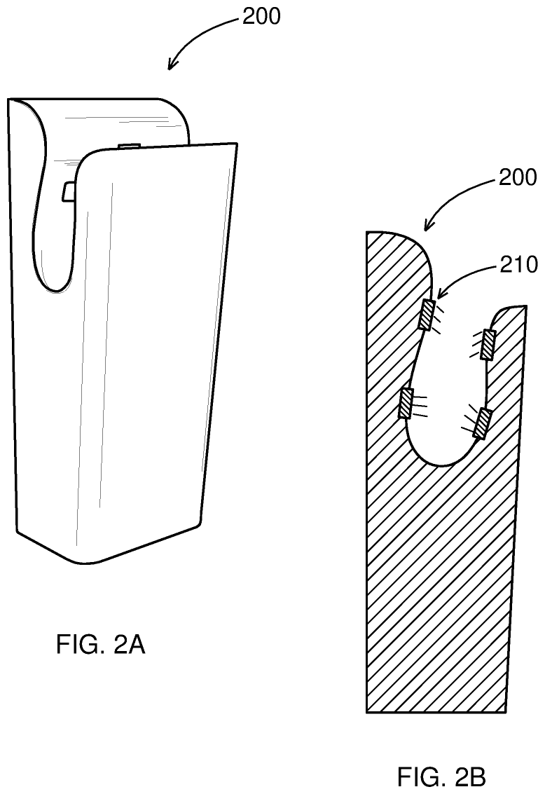

Now referring to FIGS. 2A and 2B, a hand dryer is shown. A user inserts their hands into the drying area. Sensors detect the hands and use jets of air to dry the hands. A user dries his/her hands by placing the hands below the dryers with their fingers extended. The user's fingers are typically extended straight out and held slightly apart from each other to allow for more effective drying. This creates an optimal opportunity for examining the hands and fingers.

In various exemplary embodiments, the hand dryer 200 contains one or more sensors 210. The sensors 210 are located on either side of the dryer 200 to be able to examine both the front and the back of the user's hands.

In various exemplary embodiments, the imaging sensors include one or more of infrared sensors, near infrared sensors, and visible light sensors. These sensors can detect hand size or shape, discoloration, swelling, veins, and changes over time across multiple uses. The images can also be used to identify a user and track hand appearance over time to detect trends relevant to health and wellness. In various exemplary embodiments, the various sensor arrays may be fixed or may be adapted to move to obtain more complete and accurate images.

In various exemplary embodiments, the one or more imaging sensors include one or more longwave infrared (LWIR) imaging cameras or thermal cameras. These cameras detect heat differences across the feet that can be indicative of a wide variety of health and wellness issues.

In various exemplary embodiments, the one or more imaging sensors include one or more near infrared (NIR) sensitive cameras and a source of NIR light. By illuminating the veins with NIR light, veins located just below the skin can be detected, evaluated, and tracked. Data gathered over time about veins allows one to track trends and detect changes in size and other characteristics. This information may be used to monitor indicators for potential blood clots, blood flow, and general vein health.

In various embodiments, the one or more imaging sensors include one or more visual light cameras. Computer vision may be used to process and analyze the images for indicators of many different health and wellness issues. Current images may also be compared to past images to identify changes and indicators of health and wellness issues.

In various exemplary embodiments, the one or more imaging sensors include one or more 3D cameras. 3D cameras are used to gather spatial information and data about the size and shape of the feet. This data can be used to track changes in the size and dimension of the feet indicative of health and wellness problems such as, but not limited to, swelling, water retention, blood circulation deficiencies, bunions, growths, hammer toes and other toe deformities, and arch problems.

In various exemplary embodiments, the one or more imaging sensors include one or more ultrasonic 3D imaging devices. Ultrasound technology may be used to create images of the inside of the foot.

In various exemplary embodiments, the one or more imaging sensors include one or more infrared (IR) cameras. Temperature data can be used to identify hot spots indicative of injury or developing conditions before they manifest noticeable symptoms. It can also be used to monitor general blood flow through the feet and other health and wellness indicators.

In various exemplary embodiments, the one or more sensors may comprise an array of sensors including one or more, including all possible combinations, of the sensors described above.

In various exemplary embodiments, artificial intelligence may be used to examine and analyze the data from any or all of the imaging sensors discussed herein using algorithms such as those used in computer vision and other relevant processing methods.

In various exemplary embodiments, the system includes user identification technology such as facial or hand recognition in order to identify and track the data of individual users. In some embodiments, the system includes recording or monitoring facial expressions during washing. In some embodiments, the system includes a weight scale positioned in front of the system in order to measure and track user weight over time. In various exemplary embodiments with facial recognition capability, the facial recognition system may be located in a mirror in front of the user.

In various exemplary embodiments, a weight scale is placed in front of the sink to measure the user's weight.

In an exemplary embodiment, the system includes at least one processor that receives data from the sensors. The processor is adapted to process data from the one or more sensors to create one or more images. The processor is also adapted to analyze the one or more images to detect anomalies and potential problems. The processer is also adapted to compare the one or more images processed currently with past images of the same user to detect changes over time.

In an exemplary embodiment, the system makes data gathered and processed available to the user via one or more channels, e.g., via a digital device. In a preferred embodiment, the digital device is a smart phone. In other exemplary embodiments, digital device is a computer, tablet, or other electronic device. Preferably, the system provides a report to each user of the system. In addition, a report may be provided to a healthcare provider, a care giver or a family member. Preferably, the report is prepared in such a way as to provide useful and easily understood information. Also, the report preferably includes information on trends, i.e. to indicate if conditions have gotten worse or better over time.

In preferred embodiments, the sensors are positioned so that they are directed at the hands with no other part of the user's body in the sensors' field of view. In various embodiments, additional security measures such as cryptography and image obfuscation may be used to protect the user's privacy, identity, and personal information.

In various exemplary embodiments, artificial intelligence may be used to examine and analyze the data from any or all of the imaging sensors discussed herein using algorithms such as those used in computer vision and other relevant processing methods.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

The invention has been described with reference to various specific and preferred embodiments and techniques. Nevertheless, it is understood that many variations and modifications may be made while remaining within the spirit and scope of the invention.

What is claimed is:

1. A health and wellness monitoring system comprising:
a hand washing fixture;
a sensor associated with the hand washing fixture configured to acquire data from a user's hands.

2. The system of claim 1 wherein the hand washing fixture is a faucet and wherein the sensor is located so as to acquire data from a user's hands while underneath the faucet.

3. The system of claim 2 wherein the sensor is located on and underside of the faucet.

4. The system of claim 1 wherein the handwashing fixture is a dryer and wherein the sensor is located so as to acquire data from a user's hands while using the dryer.

5. The system of claim 4 wherein the dryer is configured to direct air downward and wherein the sensor is located on an underside of the dryer.

6. The system of claim 1 comprising both a faucet and a dryer.

7. The monitoring system of claim 1 wherein the sensor comprises an imaging sensor.

8. The monitoring system of claim 1 wherein the sensor comprises one or more of ultraviolet light sensors, infrared light sensors, near infrared light sensors, and visible light sensors.

9. The monitoring system of claim 1 further comprising a processor for operating the sensor and receiving data from the sensor.

10. The monitoring system of claim 6 wherein the processor transmits health and wellness data derived from the sensor to a cloud storage.

11. The monitoring system of claim 1 wherein the sensor is located on the neck.

12. The monitoring system of claim 1 further comprising a user identification system.

13. A method of monitor health and wellness comprising:
providing a handwashing fixture;
associating a sensor with the hand washing fixture configured to acquire data from a user's hands.

14. The method of claim 13 wherein the hand washing fixture is a faucet and wherein the sensor is located so as to acquire data from a user's hands while underneath the faucet.

15. The method of claim 13 wherein the handwashing fixture is a dryer and wherein the sensor is located so as to acquire data from a user's hands while using the dryer.

16. The method of claim 13 wherein the dryer is configured to direct air downward and wherein the sensor is located on an underside of the dryer.

17. The method of claim 13 wherein the handwashing fixture comprises both a faucet and a dryer.

18. The method of claim 13 wherein the sensor comprises an imaging sensor.

19. The method of claim 13 further comprising a processor for operating the sensor and receiving data from the sensor.

20. The method of claim 13 further comprising a user identification system.
